SEQUENCE LISTING (4 sequences)

SEQ ID NO: 1 - DNA, 20 bp, artificial sequence
  M70F
gaacaatccggagttgacaa

SEQ ID NO: 2 - DNA, 20 bp, artificial sequence
  M70R
agcacgctgtcaatcatgta

SEQ ID NO: 3 - DNA, 22 bp, artificial sequence
  M22F
gctgacggctgcactgtcgggc

SEQ ID NO: 4 - DNA, 22 bp, artificial sequence
  M22R
cgttggccgggctggtttggcc

(19) United States
(12) Patent Application Publication
(10) Pub. No.: US 2020/0264193 A1
(43) Pub. Date: Aug. 20, 2020

(54) DIAGNOSTIC MARKERS FOR BOVINE TUBERCULOSIS AND USES THEREOF

(71) Applicant: INSTITUTE OF ANIMAL SCIENCES OF CHINESE ACADEMY OF AGRICULTURAL SCIENCES, BEIJING (CN)

(72) Inventors: TING XIN, BEIJING (CN); HONG JIA, BEIJING (CN); XINTAO GAO, BEIJING (CN); XIAOYU GUO, BEIJING (CN); YITONG JIANG, BEIJING (CN); HONGFEI ZHU, BEIJING (CN); SHAOHUA HOU, BEIJING (CN); WEIDONG LIN, BEIJING (CN)

(73) Assignee: INSTITUTE OF ANIMAL SCIENCES OF CHINESE ACADEMY OF AGRICULTURAL SCIENCES

(21) Appl. No.: 16/794,435

(22) Filed: Feb. 19, 2020

(30) Foreign Application Priority Data
Feb. 19, 2019 (CN) 201910121698.5

(51) Int. Cl. G01N 33/68; G01N 33/543

(52) U.S. Cl. CPC G01N 33/6869; G01N 33/54306; G01N 2800/52; G01N 2333/705; G01N 2800/12; G01N 2333/5421

(57) ABSTRACT

The present invention provides diagnostic markers for Bovine tuberculosis and uses thereof. In the present invention, markers capable of differentiating Bovine tuberculosis negative or positive are obtained by screening with untargeted proteomic techniques and verifying with targeted proteomic techniques, which are IL-8, CRP. The diagnostic markers for Bovine tuberculosis provided in the present invention can identify whether the cattles to test are Bovine tuberculosis negative or positive, and whether positive tuberculosis cattles are at discharge period of bacteria. The diagnostic markers can be used in the preparation of kits or reagents for detecting Bovine tuberculosis, thus providing new detection targets for the diagnosis of Bovine tuberculosis, which is helpful to the prompt detection and elimination of tuberculosis cattles, also ensuring the comprehensive prevention and control of Bovine tuberculosis.

### TECHNICAL FIELD

The disclosure relates to diagnostic markers for Bovine tuberculosis and their uses in the preparation of diagnostic kits for Bovine tuberculosis.

### BACKGROUND

Bovine tuberculosis is one of the chronic consumptive contagious infectious diseases primarily caused by infection with Mycobacterium bovis (M. bovis) which is a member of Mycobacterium tuberculosis comples (MTBC). Compared with Mycobacterium tuberculosis, M. bovis has a wider range of hosts, cattles are most susceptible, meanwhile it also can infect other livestocks, primates, feline, canine, as well as wild ruminants. Additionally, it may infect human by inhalation of bacterial aerosol or comsumption of unpasteurised milk, and spread between human and animals as well as from person to person. Tuberculosis cattle excretes pathogens in the form of aerosol outside the body. M. bovis-containing aerosol may adhere to meadows, sinks, etc. A healthy animal may become infected if it inhales 6 to 10 bacteria. In addition, a cow with tuberculous mastitis may release plenty of M. bovis during giving milk that is enough to pollute the total amount of milk produced from 100 healthy cows. It is demonstrated from studies that Human Immunodeficiency Virus (HIV) patients are more likely to be co-infected with M. bovis, and develop into active tuberculosis, causing M. bovis becoming more likely to be communicated to close contacts. Tuberculosis has become the most important killer of HIV patients. Therefore, Bovine tuberculosis not only harms the healthy development of aquaculture, also causes serious public health safety problems and threatens people's health and lives, so the effective prevention and control of the disease is directly related to human health.

Tuberculin skin test (TST) is one of the first methods used to diagnose Bovine tuberculosis, also is the most widely used standard method for detecting Bovine tuberculosis in the world courently, that is recommended by Office International Des Epizooties (OIE). Interferon gamma release assay (IGRA) may be recommended by OIE as the alternative detection method of tuberculin skin test for Bovine tuberculosis detection.

At present, skin test and IFN-γ release assay primarily uses bovine tuberculin (PPD-B) as the stimulus, the production process of which needs virulent strain of M. bovis, with a risk of poisoning. There are common antigens in bovine tuberculin and avian tuberculin (PPD-A), environmental mycobacteria, BCG, and there may be results of partial pseudotuberculous positive cattles. There are qualitative and quantitative difficulties due to the mixture of multiple proteins, lipids and saccharides, and it is difficult to maintain stability between batches. Recombinant M. bovis specific proteins such as CFP-10, ESAT-6 and the like have advantages of definite ingredients, ease of quality control, simple preparation and without biosafety hazards. It is demonstrated from studies that Bovine tuberculosis can be specifically detected by skin test or IFN-γ release assay with CFP-10 and ESAT-6 as the stimulus, but the sensitivity still needs to be further improved. Although many diseases can be effectively diagnosed by detecting antigens or antibodies in the blood, the serological diagnosis of tuberculosis is still a difficult point. Currently, antigens used for the serological diagnosis of tuberculosis in the world are mainly PPD, MPB70, Ag85 complex, MPB83 proteins and polypeptides and the like. Redchuk et, al. employs indirect ELISA detection method established with MPT63 and MPB83 to identify and diagnose M. bovis infection and environmental mycobacteria infection. Serological detection methods are very convenient, however, serological diagnostic methods currently established for Bovine tuberculosis all fail to achieve effective sensitivity and specificity, so they have not been recommended to use by International tuberculosis research organization. The current established skin test, IFN-γ release assay and serological detection methods all can not differentiate tuberculosis cattles at the discharge period of bacteria from those not at the discharge period of bacteria. Nested PCR detection methods can be used for detecting M. bovis pathogens in nasal secretions, alveolar lavage fluid and milk. The studies show that M. bovis pathogens can be detected in nasal swabs of 23% to 80% of tuberculosis positive cattles. However, this method has high requirements for detecting environment, technologies and personnels, so it is difficult to promote the application at grassroots level. Therefore, screening biomarkers for tuberculosis cattles, especially at the discharge period of bacteria, and establishing the relevant diagnostic methods are helpful to the prompt detection and elimination of tuberculosis cattles, also beneficial to the comprehensive prevention and control of Bovine tuberculosis.

### SUMMARY

The present invention aims to provide a diagnostic marker for Bovine tuberculosis to identify tuberculosis cattles, especially tuberculosis cattles at the discharge period of bacteria.

To achieve the above objective, a proteomic high throughput screening technology is first employed to perform an untargeted proteomic screening on plasma samples from 60 cattles including tuberculosis PCR positive cattles, tuberculosis PCR negative cattles, and tuberculosis negative cattles, discovering differences in expression levels of 223 plasma proteins between tuberculosis cattles and healthy cattles, as well as differences in expression levels of 107 plasma proteins between tuberculosis cattles at the discharge period of bacteria and tuberculosis cattles not at the discharge period of bacteria. Upon verification through targeted proteomic techniques, it is determined that 8 plasma proteins have the potential as diagnostic markers, which are monocyte differential antigen CD14, C-reactive protein (CRP), complement 6 (C6), IL-8, transfenin (TF), EGF fibrinoid extracellular matrix protein (EFEMP2), α-1-acid glycoprotein (agp), interleukin-receptor antagonist protein (IL1RN) respectively. They are screened by ELISA to finally obtain two markers relavent to the diagnosis of Bovine tuberculosis, which are IL-8 and/or CRP.

The present invention provides a use of IL-8 and/or CRP in the preparation of Bovine tuberculosis diagnostic kits.

The present invention provides a use of IL-S in the preparation of Bovine tuberculosis diagnostic kits, the use is to diagnose Bovine tuberculosis negative or positive.

In one embodiment of the present invention, it is found that when the concentration of IL-8 in plasma after PPD-B stimulation is ≥42 ng/ml, it is determined as tuberculosis positive; when the concentration of IL-8 in plasma after PPD-B stimulation is <42 ng/ml, it is determined as tuberculosis negative. Different sample sizes may affect the accuracy of detection threshold, however, upon careful screening and statistics, the inventors found that the average concentration of IL-8 in plasma of tuberculosis cattles after PPD-B stimulation is greater than five times of that in tuberculosis negative cattles, significantly higher than in negative cattles. It is also found in the invention that after stimulation with other well-known stimulus, the concentration of IL-S in plasma is significantly different from the concentration values in tuberculosis negative and positive cattles, and similar to the situation after PPD-B stimulation.

Preferably, the average concentration of IL-8 in plasma of tuberculosis cattles is greater than six times of that in tuberculosis negative cattles, significantly higher than in negative cattles.

The present invention provides a use of CRP in the preparation of Bovine tuberculosis diagnostic kits, the use is to diagnose tuberculosis cattles at the discharge period of bacteria

It is found in embodiments of the present invention that for tuberculosis positive cattles, when the concentration of CRP in plasma after PPD-B stimulation is >790 ng/ml, it is determined as cattles at the discharge period of bacteria, while when the concentration of CRP in plasma after PPD-B stimulation is <790 ng/ml, it is determined as cattles not at the discharge period of bacteria. When the concentration of IL-8 in plasma is >42 ng/ml, and the concentration of CRP in plasma is <790 ng/ml, it is determined as tuberculosis positive cattles not at the discharge period of bacteria. It is also found in the invention that after stimulation with other well-known stimulus, the concentration of CRP in plasma is significantly different from the concentration values in tuberculosis positive cattles at the discharge period of bacteria and not at the discharge period of bacteria, and similar to the situation after PPD-B stimulation.

Meanwhile, upon careful screening and statistics, the inventors further found that the concentration of CRP in tuberculosis cattles at the discharge period of bacteria after PPD-B stimulation is greater than three times of that in tuberculosis cattles not at the discharge period of bacteria and that in negative cattles, significantly higher than in tuberculosis cattles not at the discharge period of bacteria and in negative cattles.

Preferably, the concentration of CRP in tuberculosis cattles at the discharge period of bacteria after stimulus stimulation is greater than four times of that in tuberculosis cattles not at the discharge period of bacteria and that in negative cattles, significantly higher than in tuberculosis cattles not at the discharge period of bacteria and in negative cattles.

The present invention provides a use of IL-8 and CRP in the preparation of diagnostic kits used for tuberculosis cattles at the discharge period of bacteria and tuberculosis cattles not at the discharge period of bacteria.

The present invention provides a use of CRP in the preparation of diagnostic kits used for tuberculosis cattles at the discharge period of bacteria and tuberculosis cattles not at the discharge period of bacteria.

In the above uses of the invention, the kits are ELISA kits.

Further, the ELISA kits also contain stimulus, the stimulus is bovine tuberculin, avian tuberculin, CFP-10, ESAT-6 or PBS.

The present invention provides a diagnostic kit used for tuberculosis cattles at the discharge period of bacteria and tuberculosis cattles not at the discharge period of bacteria, the kit contains detection reagents for detecting the expression level of IL-8 and/or CRP.

The present invention has the following beneficial effects: molecular markers relevant to Bovine tuberculosis infection are screened with an untargeted proteomic technique, and verified by an targeted proteomic technique and ELISA processes, from which the most potential molecular markers for Bovine tuberculosis are obtained that are cytokines IL-8 and CRP, and the expression levels of IL-8 and CRP are markedly higher than those of IFN-γ, IP-10, and IL-17A, and have a good correlation with the expression level of IFN-γ, thus having an advantage of being the molecular markers for Bovine tuberculosis. The present invention first discovers and verifies that IL-8 and CRP can be used as diagnostic markers of Bovine tuberculosis, and discovers that they can differentiate tuberculosis cattles at the discharge period of bacteria from tuberculosis cattles not at the discharge period of bacteria.

The present invention constructs an IL-8, CRP sandwich ELISA kit for Bovine tuberculosis. The kit is used to detect the expression levels of IL-8, CRP in plasma after stimulation with M. bovis specific antigen (PPD-B/CE stimulation), the expression level of IL-8 in tuberculosis positive cattles is significantly higher than that in healthy cattles (the concentration of IL-8 in plasma is greater than five times of that in tuberculosis negative cattles), and the expression level of CRP in cattles at the discharge period of bacteria is significantly higher than that in cattles not at the discharge period of bacteria and that in healthy cattles (the concentration of CRP in plasma is greater than three times of that in tuberculosis cattles not at the discharge period of bacteria and that in negative cattles). This detection method may improve the diagnostic efficiency of Bovine tuberculosis, and may differentiate PCR positive and negative cattles, which is helpful to the prompt detection and elimination of cattles at the discharge period of bacteria, also beneficial to the prevention and control and decontamination of Bovine tuberculosis in China.

### BRIEF DESCRIPTION OF THE DRAWINGS

FIG. 1 is a diagram showing effects after extracting high-abundant proteins in serum and plasma samples detected by SDS-PAGE. Lane M: protein molecular weight standard; Lane 1: bTB_{PCR-P} P1, after extracting peak-abundant proteins; Lane 2: bTB_{PCR-P} P1, before extracting high-abundant proteins; Lane 3: bTB_{PCR-P} P2, after extracting high-abundant proteins; Lane 4: bTB_{PCR-P} P2, before extracting high-abundant proteins; Lane 5: bTB_{PCR-P} P1, after extracting high-abundant proteins; Lane 6: bTB_{PCR-N} P1, before extracting high-abundant proteins; Lane 7: bTB_{PCR-N} P2, after extracting high-abundant proteins; Lane 8: bTB_{PCR-N} P2, before extracting high-abundant proteins; Lane 9: NC P1, after extracting high-abundant proteins; Lane 10: NC P1, before extracting high-abundant proteins; Lane 11: NC P2, after extracting high-abundant proteins; Lane 12: NC P2, before extracting high-abundant proteins.

FIG. 2 is a diagram showing the expression level of TF in plasma after PPD-B stimulation.

FIG. 3 is a diagram showing the expression level of agp in plasma after PPD-B stimulation.

FIG. 4 is a diagram showing the expression level of IL-8 in plasma after PPD-B stimulation.

FIG. 5 is a diagram showing the expression level of IP-10 in plasma after PPD-B stimulation.

FIG. 6 is a diagram showing the expression level of IL-17 in plasma after PPD-B stimulation.

FIG. 7 is a diagram comparing the concentrations of IL-8, IFN-γ, IP-10 and IL-17 in plasma after PPD-B stimulation.

FIG. 8 is a diagram showing the expression level of CRP in plasma after PPD-B stimulation.

FIG. 9 is a diagram showing the expression level of IL-8 after CFP-10-ESAT-6 stimulation.

FIG. 10 is a diagram showing the expression level of IP-10 after CFP-10-ESAT-6 stimulation.

FIG. 11 is a diagram showing the expression level of IL-17 after CFP-10-ESAT-6 stimulation.

### DESCRIPTION OF THE EMBODIMENTS

The present invention will be further explained below in conjuction with specific embodiments. These embodiments are only used to illustrate the invention, while not limit the scope of the invention. Bovine tuberculin (PPD-B) and purified avine tuberculin (PPD-A) are purchased from The Sixth Factory of Harbin Pharmaceutical Group Co., Ltd. Recombinant proteins ESAT-6-CFP-10, CFP-10, and ESAT-6 are prepared by Tuberculosis and Animal Epidemics Diagnosis Laboratory of Beijing Animal Husbandry and Veterinary Research Institute in Chinese Academy of Agricultural Sciences, and have been published in the document Xin T, Jia H, Ding J, Li P, Yang H, Hou S, Yuan W, Guo X, Wang H, Liang Q, Li M, Wang B, Zhu H.2013. Assessment of a protein cocktail-based skin test for Bovine tuberculosis in a double-blind field test in cattle. Clin Vaccine Immunol 20:482-90.

### Embodiment 1 Detection and Collection of Clinical Samples Involved in the Present Invention

1. Tuberculin Skin Test:

The tuberculin skin test is performed according to Diagnostic Criteria for Bovine Tuberculosis (GB/T 18645-2002). The cattle is shaved at ⅓ of the neck, and intradermally injected with 0.1 mL purified bovine tuberculin (PPD-B, 250 IU/dose). Skin thicknesses at the injection sites are measured by the same operator with a vernier caliper before injection and 72 h after injection respectively, and the skin thickness difference is calculated. When the skin thickness difference is greater than or equal to 4 mm, the cattle is tuberculosis positive; when the skin thickness difference is smaller than 2 mm, it is determined as tuberculosis negative; when the skin thickness difference is between 2 mm and 4 mm, it is determined as suspected, the skin test needs to be performed once again 60 days after the first detection, and if the skin thickness difference is greater than or equal to 2 mm at the second detection, then it is determined as tuberculosis positive.

2. CFP-10/ESAT-6/TB10.4-Based Skin Test:

The cattle is shaved at ⅓ of the neck, and intradermally injected with 0.1 mL recombinant protein CFP-10/ESAT-6/TB10.4 (the concentration of the recombinant protein is 0.5 mg/ml, the ratio of CFP-10, ESAT-6 and TB10.4 is 1:1:1). Skin thicknesses at the injection site are measured by the same operator with a vernier caliper before injection and 72 h after injection respectively, and the skin thickness difference is calculated. When the skin thickness difference is greater than or equal to 2 mm, the cattle is tuberculosis positive; when the skin thickness difference is smaller than 2 mm, then it is determined as tuberculosis negative.

3. Interferon Gamma Release Assay:

10 ml of heparin lithium anti-coagulant blood is harvested and transferred to the laboratory at room temperature (22±4° C.) within 16 hours. The anti-coagulant blood is firstly added onto a 24-well tissue culture plate at 1.5 ml per well. Each well is aseptically added with purified bovine tuberculin (PPD-B), purified avian tuberculin (PPD-A), and PBS each 100 μl, blended with shaking and then incubated in a incubator at 37° C. and C02 for 20 to 24 hours. 200 μl of upper plasma is sucked up carefully and transferred into a 1.5 ml centrifuge tube, ready for use (the plasma can be stored at 2 to 8° C. for 7 days, and can be stored at −20° C. for several months). Following the instruction of bovine IFN-γ detection kit (purchased from Prionics Co.), OD_{450 nm} values for PPD-B, PPD-A and PBS stimulated samples are marked as OD_{450 nm (PPD-B)}, OD_{450 nm (PPD-A)}, OD_{450 nm (PBS)}, respectively. When OD_{450 nm (PPD-B)}−OD_{450 nm (PPD-A)}≥0.1 and OD_{450 nm (PPD-B)}−OD_{450 nm (PBS)}≥0.1, it is determined as Bovine tuberculosis positive, while when OD_{450 nm (PPD-B)}−OD_{450 nm (PPD-A)}<0.1 or OD_{450 nm (PPD-B)}−OD_{450 nm (PBS)}<0.1, it is determined as Bovine tuberculosis negative.

4. CFP-10-ESAT-6-Based Interferon Gamma Release Assay:

ml of heparin lithium anti-coagulant blood is harvested and transferred to the laboratory at room temperature (22±4° C.) within 16 hours. The anti-coagulant blood is firstly added onto 24-well tissue culture plates at 1.5 ml per well. Each well is aseptically added with recombinant protein ESAT-6-CFP-10 (CE, 20 μg/ml, endotoxin<10 EU/mg) and PBS each 100 μl, blended with shaking and then incubated in a incubator at 37° C. and CO₂ for 20 to 24 hours. 200 pd of upper plasma is sucked up carefully and transferred into a 1.5 ml centrifuge tube, ready for use (the plasma can be stored at 2 to 8° C. for 7 days, and can be stored at −20° C. for several months). Following the instruction of bovine IFN-γ detection kit (purchased from Prionics Co.), OD_{450 nm} values for ESAT-6-CFP-10 and PBS stimulated samples are marked as OD_{450 nm (CE)} and OD_{450 nm (PBS)}, respectively. When OD_{450 nm (CE)}−OD_{450 nm (PBS)}≥0.1, it is determined as Bovine tuberculosis positive, while when OD_{450 nm (CE)}−OD_{450 nm (PBS)}<0.1, it is determined as Bovine tuberculosis negative.

5. Nested PCR Detection on Nasal Swab Secretion:

To a sterile tube is charged with 2 to 4 ml of sterile PBS. A flocking swab is stretched into the nasal cavity of a cattle and turned for 5 to 8 circles, and then immediately put into the sterile tube, with the tip of the swab being immerged in PBS. The tip of the swab is broken off, the lid is screwed on tightly, and the tube is kept at low temperature. It is centrifuged at 13000 r/min for 15 mins at 4° C., discarding the supernatant. A genome is extracted with a bacterial genome extraction kit (purchased from TAKARA Co.) and cryopreserved at −20° C. ready for use. With the extracted genomic DNA as the template, a specific target sequence (372 bp) of mpb70 gene in Mycobacterium tuberculosis comples is amplified with M70F and M70R, the reaction system is: DDW 19 μL, 2×PCR Mix 25 μL, M70F (10 μM) 2 μL, M70R (10 μM) 2 μL, genomic DNA template 2 μL.

The reaction conditions of PCR amplification are: predegeneration at 94° C. for 5 mins; degeneration at 94° C. for 45 s, annealing at 60° C. for 30 s, extension at 72° C. for 45 s, 30 cycles; and extension at 72° C. for additional 5 mins. 1 μl of PCR products are respectively taken as the template of the second run of PCR reaction, of which the 50 μL PCR reaction system is: DDW 20 μL, 2×PCR Mix 25 μL, M22F (10 μM) 2 μL, M22R (10 μM) 2 μL, and PCR product template 1 μL. The second run of PCR is amplified with touch down cyclic parameters.

TABLE 1
Names, Sequences and Amplification Pro-
duct Sizes of Nested PCR Primers

| | | Ampli- |
|---|---|---|
| | | fied |
| Primer | | Fragment |
| Name | Primer Sequence | (bp) |
| M70F | GAACAATCCGGAGTTGACAA | 372 |
| | (SEQ ID NO. 1) | |
| M70R | AGCACGCTGTCAATCATGTA | |
| | (SEQ ID NO. 2) | |
| M22F | GCTGACGGCTGCACTGTCGGGC | 208 |
| | (SEQ ID NO. 3) | |
| M22R | CGTTGGCCGGGCTGGTTTGGCC | |
| | (SEQ ID NO. 4) | |

6. Detection is performed using the method of the above steps 1-4 to screen tuberculosis positive cattles and tuberculosis negative cattles, and tuberculosis cattles are grouped into PCR positive cattles and PCR negative cattles using the method of the above step 5.

TABLE 2
Clinical Sample Grouping

| | PCR Positive | PCR Negative | Tuberculosis |
|---|---|---|---|
| | Tuberculosis | Tuberculosis | Negative |
| | Cattle | Cattle | Cattle |
| Detection Method | (bTB_{PCR-P}) | (bTB_{PCR-N}) | (NC) |
| Tuberculin Skin Test | + | + | − |
| CFP-10/ESAT-6/ | + | + | − |
| TB10.4-based Skin Test | | | |
| Interferon Gamma | + | + | − |
| Release Assay | | | |
| CFP-10-ESAT-6-based | + | + | − |
| Interferon Gamma | | | |
| Release Assay | | | |
| Nested PCR Detection | + | − | − |
| on Nasal Swab Secretion | | | |

7. Sample Collection and Preparation:

10 ml of venous blood is harvested from a cattle aseptically, and injected into a heparin lithium anticoagulant blood collection tube (10 ml). The anti-coagulant blood is added onto a 24-well tissue culture plate at 1.5 ml/well. Each well is aseptically added with purified bovine tuberculin (PPD-B), purified avian tuberculin (PPD-A), PBS, and recombinant protein ESAT-6-CFP-10 (CE, 20 μg/ml, endotoxin<10 EU/mg) each 100 μl, blended with shaking and then incubated in a incubator at 37° C. and CO₂ for 20 to 24 hours. The upper plasma is sucked up carefully, transferred into a 1.5 ml centrifuge tube, and cryopreserved at −80° C. ready for use.

### Embodiment 2 Screening on Molecular Markers

1. Sample pretreatment: 20 PCR positive tuberculosis cattles (bTB_{PCR-P}), 20 PCR negative tuberculosis cattles (bTB_{PCR-N}) and 20 tuberculosis negative cattles (NC) identified in Embodiment 1 are randomly screened. Take the bTB_{PCR-P} group as an example, every 10 plasma samples are mixed at equal volumes, becoming two biological repetitive plasma mixed samples (bTB_{PCR-P} P1 is the plasma mixed sample of cattles Nos. 1-10, and bTB_{PCR-P} P2 is the plasma mixed sample of cattles Nos. 11-20). Following this method, a plasma mixed sample of bTB_{PCR-N} (bTB_{PCR-N} P1 and bTB_{PCR-N} P2) and NC group (NC P1 and NC P2) is prepared.

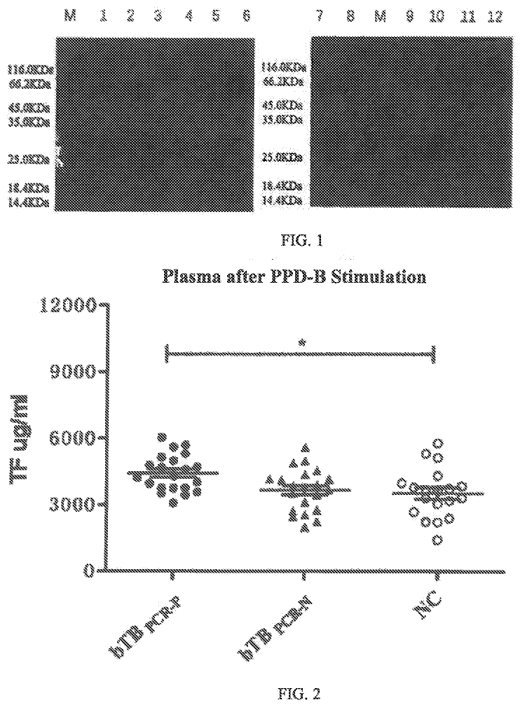

2. Extraction of high-abundant proteins: As IgG, BSA, etc. in plasma account for more than 85% of the total proteins, which may affect the detection of low-abundant proteins, so high-abundant protein extraction kits (purchased from Bio-Rad Co.) are used to extract high-abundant proteins from plasma samples. The extraction effects of high-abundant proteins are detected with SDS-PAGE, and a Bradford protein quantification kit is used to detect the concentration of proteins. It is shown from the results that high-abundant proteins are successfully extracted from 6 plasma samples (bTB_{PCR-P} P1, bTB_{PCR-P} P2, bTB_{PCR-N} P1, bTB_{PCR-N} P2, NC P1 and NC P2) (as shown in FIG. 1), and the concentrations of proteins are all greater than 2 mg/ml, which can be detected with mass spectrometry.

3. Enzyme digestion and desalination of proteins: 100 μg proteins are taken and adjusted the volume to 100 μl with a solution of triethylammonium bicarbonate (TEAB), then diluted with 500 μl of NH₄HCO₃ at 50 mM, and digested with 2 μg trypsin solution at 37° C. overnight, and then acidified with an equal volume of 0.1% formic acid (FA); a Strata-X C18 column is taken out and activated with 1 ml methanol, and equilibrated with 1 ml of 0.1% FA; the above acidified enzymatic hydrolyzates are added into the Strata-X C18 column, and filtered serially for 3 times, then the Strata-X C18 column is washed with 0.1% FA+5% acetonitrile serially for 2 times; the Strata-X C18 column is eluted by adding 1 ml of 0.1% FA+80% acetonitrile for one time, and 1 ml of liquid is collected into a new centrifugal tube; the liquid is freezed and dried, and then redissolved with 20 μl of TEAB at 0.5 M.

4. iTRAQ LC-MS/MS Detection: Following the instruction, 8-plex markers are employed, which are specifically as follows: bTB_{PCR-P} Group: 113, 114; bTB_{PCR-N}: 115, 116; NC: 117, 118. After being marked, 6 plasma samples to be detected are mixed at equal parts respectively. The mixed samples are divided into 16 components, detected by mass spectrometry with AB SCIEX nanoLC_MS/MS (Triple TOF 5600plus), and data search and identification are performed with Proteinpilot™ V4.5. Proteins of which the unused score≥1.3 (i.e, level of confidence above 95%), containing at least one unique peptide segment, are considered as confident proteins. The average of ratios between every two replicate samples is normalized by the median to be the difference factor of samples to be compared, and the minimum value in p-values of one-sample Student's t test between every two replicate samples is utilized as the significance difference test p value among samples to be compared. When the difference factor reaches 1.5 folds and above (that is, up_regulate≥1.5 and down_regulate≤0.67), and its p-value≤0.05, it is considered as significantly differential protein. It is shown from the results that there are 719 proteins identified from the plasma sample, of which 531 proteins have been identified more than two peptide segments. According to the multiples and functions of identified differential proteins, 15 plasma proteins are screened to be identified with parallel reaction monitoring (PRM) (see Table 3). According to the results of iTRAQ and PRM detection, proteins with consistent results which can be used for the diagnosis of Bovine tuberculosis are screened for ELISA verification.

TABLE 3
Differential proteins in plasma after PPD-B stimulation idendified by iTRAQ and PRM

| | | Fold-changes in iTRAQ | | | | Fold-changes in PRM | | | |
|---|---|---|---|---|---|---|---|---|---|
| Protein | | bTB_{PCR-P}/ | bTB_{PCR-N}/ | bTB/ | bTB_{PCR-P}/ | bTB_{PCR-P}/ | bTB_{PCR-N}/ | bTB/ | bTB_{PCR-P}/ |
| ID | Name | NC | NC | NC | bTB_{PCR-N} | NC | NC | NC | bTB_{PCR-N} |
| A8DBT6 | Monocyte | 5.49 | 4.39 | 4.67 | — | 1.8 | 1.67 | 1.74 | 1.08 |
| | differential | | | | | | | | |
| | antigen CD14 | | | | | | | | |
| C4T8B4 | C-Reactive | — | 0.08 | 0.41 | 11.36 | 1.67 | 0.57 | 1.12 | 2.92 |
| | Protein (CRP) | | | | | | | | |
| F1MM86 | Complement 6 | 5.87 | 4.23 | 4.78 | 1.63 | 1.2 | 1.2 | 1.2 | 1 |
| F1MMK9 | Protein AMBP | 4.14 | 2.71 | 3.23 | — | 1.19 | 1 | 1.1 | 1.18 |
| | (AMBP) | | | | | | | | |
| G3LUN8 | IL-8 | 9.98 | 8.59 | 8.77 | — | 2.81 | 2.42 | 2.62 | 1.16 |
| G3X6N3 | Transferrin | 11.47 | 4.42 | 7.51 | 8.96 | 2.02 | 1.35 | 1.68 | 1.5 |
| | (TF) | | | | | | | | |
| G3X6Y4 | Osteomodulin | 8.87 | 12.35 | 9.99 | — | — | — | — | — |
| | (OMD) | | | | | | | | |
| O02659 | Mannose | 6.97 | 3.46 | 4.93 | 2.34 | — | — | — | — |
| | binding | | | | | | | | |
| | protein C | | | | | | | | |
| | (MBL) | | | | | | | | |
| O77482 | Interleukin- | 5.4 | 6.79 | 5.76 | — | — | — | — | — |
| | receptor | | | | | | | | |
| | antagonist | | | | | | | | |
| | protein | | | | | | | | |
| | (IL1RN) | | | | | | | | |
| P07224 | Protein S | 5.86 | — | 4.71 | 1.52 | — | — | — | — |
| | (PROS) | | | | | | | | |
| Q011G2 | EGF fibrinoid | 12.56 | 11.94 | 11.58 | — | 1.53 | 2.56 | 2.04 | 0.6 |
| | extracellular | | | | | | | | |
| | matrix protein | | | | | | | | |
| | (EFEMP2) | | | | | | | | |
| Q2KIF2 | Interieukin- | 7.96 | — | 6.2 | 2.49 | 1.26 | 1.2 | 1.23 | 1.05 |
| | receptor | | | | | | | | |
| | antagonist | | | | | | | | |
| | protein | | | | | | | | |
| | (IL1RN) | | | | | | | | |
| Q2KIX7 | Protein HP-25 | 9.73 | 3.1 | 6.06 | 10.07 | — | — | — | — |
| | homolog 1 | | | | | | | | |
| Q5E9C0 | Ras | 0.33 | — | — | 0.23 | 0.2 | 0.68 | 0.44 | 0.3 |
| | Suppressor | | | | | | | | |
| | Protein 1 | | | | | | | | |
| | (RSU1) | | | | | | | | |
| Q5GN72 | α-1-acid | 12.68 | 4.2 | 7.97 | 11.84 | 1.32 | 1.32 | 1.32 | 1 |
| | glycoprotein | | | | | | | | |
| | (agp) | | | | | | | | |

### Embodiment 3 Verification on Molecular Markers of the Invention

1. PRM identification of differential proteins: Samples are treated following the process of Embodiment 2. Upon DDA detection, a targeted analytic strategy of mass spectrometry PRM is utilized to add the peptide segments of the identified target proteins into the inclusion list of the mass spectrometry acquisition method, allowing the mass spectrometry to collect data against these specific peptide segments, and performing relative quantitative analysis by extracting fragment ion information (results see Table 4). According to iTRAQ and PRM identification results (see Table 3), the contents of IL-8, agp and TF in plasma of tuberculosis cattles are more than 1.3 times of those in plasma of healthy cattles, while the content of CRP in plasma of tuberculosis cattles at the discharge period of bacteria is more than 2 times of that in plasma of tuberculosis cattles not at the discharge period of bacteria, and the detection results between iTRAQ and PRM are coincident. Therefore, plasma proteins IL-8, CRP, agp, and TF are screened, together with IL-17A and IP-10 reported in literature, to be detected by an ELISA method.

2. ELISA Verification of molecular markers: 21 PCR positive tuberculosis cattles (bTB_{PCR-P}), 21 PCR negative tuberculosis cattles (bTB_{PCR-N}) and 19 tuberculosis negative cattles (NC) are randomly screened utilizing the method of steps 1-5 in Embodiment 1, and plasma 'samples are prepared for each cattle following the method of step 7 in Embodiment 1.

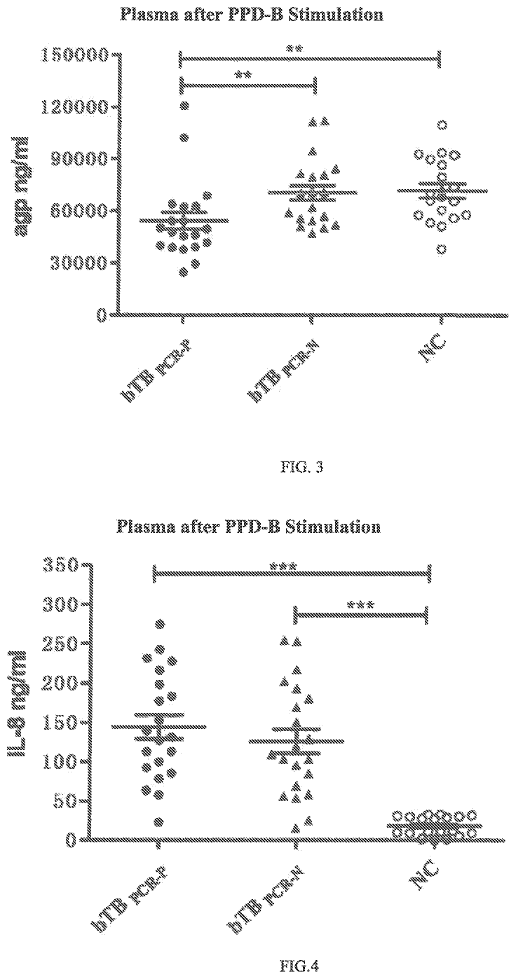

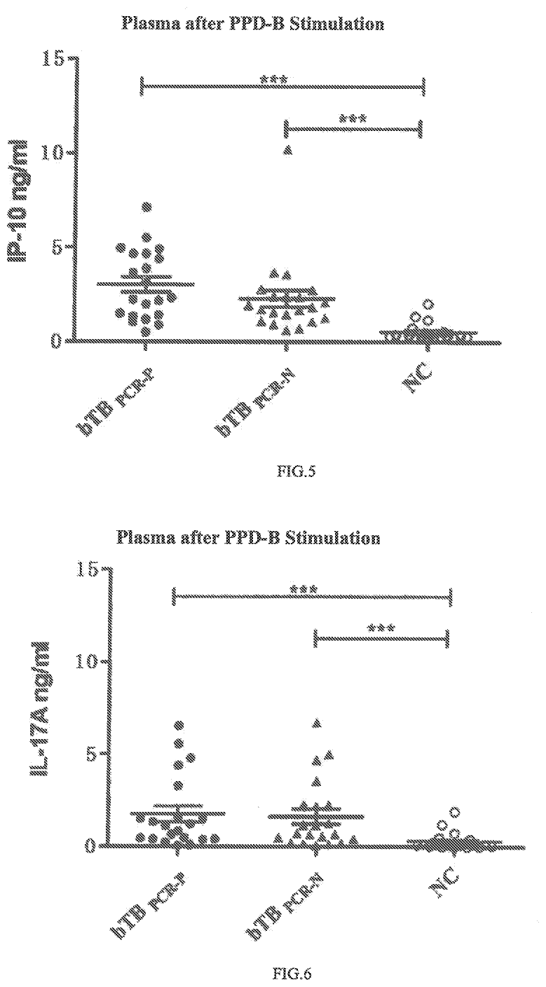

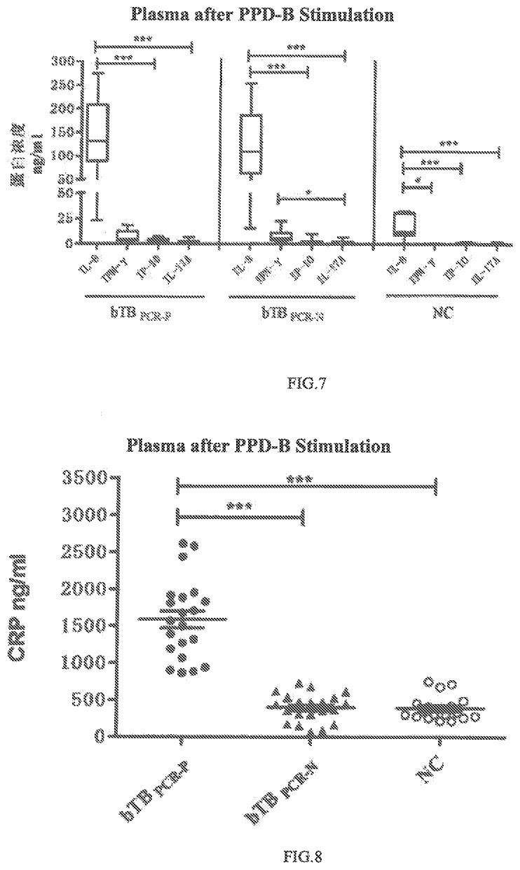

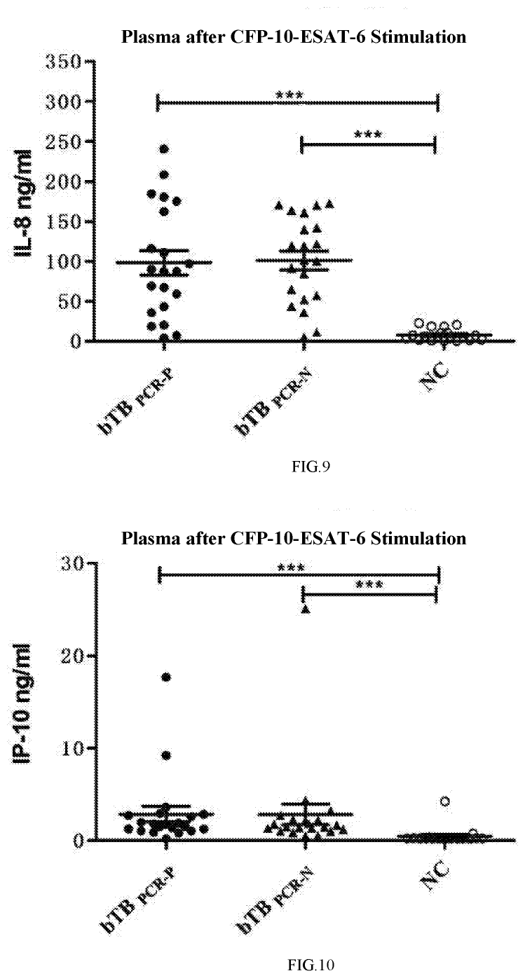

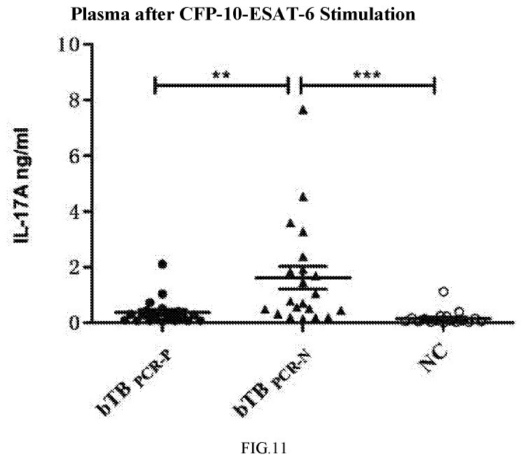

A commercial ELISA kit is used to detect Serotransferrin (TF), Alpha-1-acid glycoprotein (agp), C-reactive protein (Pentaxin, CRP), IL-8, IL-17 and IP-10 in plasma. It is shown from the results that after PPD-B stimulation, TF in plasma of bTBPcR-p Group is significantly higher than that of NC Group, while the level of TF in plasma of bTB_{PCR-N} Group is similar to that of NC Group (see FIG. 2); agp in plasma of bTB_{PCR-P} Group is significantly lower than those in bTB_{PCR-N} Group and NC Group while the level of agp in plasma of bTB_{PCR-N} Group is similar to that of NC Group (see FIG. 3). IL-8, IP-10 and IL-17 in plasma of bTB_{PCR-P} Group and bTB_{PCR-N} Group are significantly higher than that of NC Group (see FIGS. 4-6), and the concentration of IL-8 is significantly higher than IP-10 and IL-17 (see FIG. 7), while CRP in plasma of bTB_{PCR-P} Group is significantly higher than those of bTB_{PCR-N} Group and NC Group (see FIG. 8). It can be seen from this, after PPD-B stimulation, IL-8, IP-10 and IL-17 in plasma all have the potentials to differentiate tuberculosis cattles from uninfected cattles, and the concentration of IL-8 is higher, with its detection potential superior to those of IP-10 and IL-17. At the same time, the levels of IL-8, IP-10 and IL-17 in plasma after M. bovis antigen CFP-10-ESAT-6 stimulation are detected. It is found that after CFP-10-ESAT-6 stimulation, IL-8 and IP-10 in plasma of bTB_{PCR-P} Group and bTB_{PCR-N} Group are significantly higher than those in NC Group (see FIGS. 9-10), IL-17 in plasma of bTB_{PCR-N} Group is significantly higher than those in bTB_{PCR-P} Group and NC Group (see FIG. 11). Therefore, when specific antigen CFP-10-ESAT-6 is used as the stimulation antigen, IL-8 and IP-10 are superior to IL-17, and because the concentration of IL-8 is higher than that of IP-10, so IL-8 has an advantage of being a detection marker for Bovine tuberculosis.

It is shown from the above results that after PPD-B and CFP-10-ESAT-6 stimulation, IL-8 in plasma is capable of differentiating tuberculosis cattles from negative cattles, while after PPD-B stimulation, CRP is capable of differentiating tuberculosis cattles at the discharge period of bacteria from tuberculosis cattles not at the discharge period of bacteria. Therefore, M. bovis specific antigen (PPD-B, CE) induced IL-8 and CRP can be used for the diagnosis of Bovine tuberculosis, and can be used for differentiating tuberculosis cattles at the discharge period of bacteria from those not at the discharge period of bacteria.

3. Determination of Cutoff Values in Detecting Bovine Tuberculosis with IL-8, CRP Induced by PPD-B

Skin test and interferon gamma release assay are the detection methods for Bovine tuberculosis recommended by OIE, while it has reported in recent researches that IL-17A and IP-10 also have the potential as the diagnostic markers for Bovine tuberculosis. Therefore, in the present invention, a tuberculin skin test, a CFP-10/ESAT-6/TB10.4-based skin test, an interferon gamma release assay, a CFP-10-ESAT-6-based interferon gamma release assay and a nested PCR detection method of nasal swab secretion are utilized to screen 21 PCR positive tuberculosis cattles (bTB_{PCR-P}), 21 PCR negative tuberculosis cattles (bTB_{PCR-N}), and 19 tuberculosis negative cattles.

10 ml of venous blood is harvested from a cattle aseptically, and injected into a heparin lithium anticoagulant blood collection tube (10 ml). The anti-coagulant blood is added onto a 24-well tissue culture plate at 1.5 ml/well. Each well is aseptically added with purified bovine tuberculin (PPD-B), and CFP-10-ESAT-6 each 100 μl, blended with shaking and then incubated in a incubator at 37° C. and CO₂ for 20 to 24 hours. The upper plasma is sucked up carefully, transferred into a 1.5 ml centrifuge tube, and the concentrations of IFN-γ, IL-8, CRP, IP-10 and IL-17 in plasma are detected. Receiver operating characteristic curve is used for analyzing IL-8, IP-10 and IL-17 to differentiate cutoff values of tuberculosis positive cattles from those of negative cattles (comprising 42 tuberculosis positive cattles and 19 negative cattles), and CRP is analyzed to differentiate cutoff values of tuberculosis cattles at the discharge period of bacteria from those not at the discharge period of bacteria (comprising 21 tuberculosis cattles at the discharge period of bacteria and 21 cattles not at the discharge period of bacteria).

It is shown from the results that, when differentiating tuberculosis positive cattles from negative cattles with PPD-B as the stimulus, AUC of IL-8 is higher than those of IP-10 and IL-17, and when a specificity of 100% is selected, the sensitivity of detection can up to 96.62%, while when the specificities of IP-10 and IL-17 are 100%, the sensitivities of detection are only 52.38% and 28.57%, respectively. When CFP-10-ESAT-6 is used as the stimulus, AUC of IL-8 is 0.9561, the specificity is 100%, the sensitivity is 85.71%, and the detection effect is inferior to the detection effect with PPD-B as the stimulus. When differentiating tuberculosis cattles at the discharge period of bacteria from those not at the discharge period of bacteria, AUC of CRP is 1, and when the specificity is 100%, the sensitivity of detection can up to 100%. Therefore, IL-8 and CRP induced with PPD-B have more potential as molecular markers for Bovine tuberculosis than IL-8 induced with IP-10, IL-17 as well as CFP-10-ESAT-6.

TABLE 4
Determination of cutoff values

| | | Area | | 95% | | 95% | |
|---|---|---|---|---|---|---|---|
| | | Under | | Confidence | | Confidence | Cut-off |
| | | Curve | Sensitivity | Interval | Specificity | Interval | Values |
| Functions | Detection | (AUC) | (%) | (%) | (%) | (%) | (ng/ml) |
| Differentiating | PPD-B-induced | 0.9662 | 92.86 | 80.52-98.50 | 94.74 | 73.97-99.87 | >32.57 |
| tuberculosis | IL-8 | | 92.86 | 80.52-98.50 | 100 | 82.35-100.0 | >43.05 |
| positive | CFP-10-ESAT-6- | 0.9561 | 85.71 | 71.46-94.57 | 94.74 | 73.97-99.87 | >21.9 |
| cattles from | induced IL-8 | | 85.71 | 71.46-94.57 | 100.0 | 82.35-100.0 | >29.42 |
| negative | PPD-B-induced | 0.9500 | 52.38 | 36.42-68.00 | 95 | 75.13-99.87 | >1.992 |
| cattles | IP10 | | 52.38 | 36.42-68.00 | 100 | 83.16-100.0 | >2.045 |
| | PPD-B-induced | 0.8464 | 28.57 | 15.72-44.58 | 95 | 75.13-99.87 | >1.767 |
| | IL-17A | | 28.57 | 15.72-44.58 | 100 | 83.16-100.0 | >2.061 |
| Differentiating | PPD-B-stimulated | 1.000 | 100 | 83.89-100.0 | 95.24 | 76.18-99.88 | >701.3 |
| tuberculosis | CRP | | 100 | 83.89-100.0 | 100 | 83.89-100.0 | >794.8 |
| cattles at the | | | | | | | |
| discharge | | | | | | | |
| period of | | | | | | | |
| bacteria from | | | | | | | |
| tuberculosis | | | | | | | |
| cattles not at | | | | | | | |
| the discharge | | | | | | | |
| period of | | | | | | | |
| bacteria | | | | | | | |

4. Correlation between IL-8, CRP and IFN-γ, IP-10, IL-17A: A spearman r method is utilized to detect the correlation of expression levels of cytokines. It is shown from the results that the expression level of IL-8 is significantly higher than those of IFN-γ, IP-10 and IL-17 (see FIG. 7), and its correlation with IFN-γ is the highest, the correlation coefficient is greater than 0.75 (Table 5), indicating that IL-8 is more suitable to be detection markers for Bovine tuberculosis compared with IP-10 and IL-17. CRP is used to differentiate tuberculosis cattles at the discharge period of bacteria from tuberculosis cattles not at the discharge period of bacteria, its correlation with IL-8, IFN-γ, IP-10 and IL-17 is lower than that of other factors, while in significant positive correlation with IL-8 and IP-10.

TABLE 5
Correlation analysis on expression levels of cytokines

| | Correlation coefficient r of plama after PPD-B stimulation | | | | |
|---|---|---|---|---|---|
| Cytokines | IL-8 | IFN-γ | IP-10 | IL-17A | CRP |
| IL-8 | | 0.75* | 0.63* | 0.54* | 0.38 |
| IFN-γ | 0.75* | | 0.63* | 0.61* | 0.24 |
| IP-10 | 0.63* | 0.63* | | 0.47* | 0.43 |
| IL-17A | 0.54* | 0.61* | 0.47* | | 0.19 |
| CRP | 0.38* | 0.24 | 0.43* | 0.19 | |

*p < 0.05

### Embodiment 4 Construction of IL-8, CRP Detection Kit for Bovine Tuberculosis of the Invention

1. Sample Collection and Preparation:

10 ml of venous blood is harvested from a cattle aseptically, and injected into a heparin lithium anticoagulant blood collection tube, which is inverted gently for 3-5 times, and sent back to the laboratory at room temperature for 20 hours. The anti-coagulant blood is added onto a 24-well tissue culture plate at 1.5 ml/well. Each well is aseptically added with 100 μl PPD-, blended with shaking gently and then incubated in a incubator at 37° C. and CO₂ for 20 to 24 hours. The upper plasma is sucked up carefully, transferred into a 1.5 ml centrifuge tube, and cryopreserved at −80° C. ready for use.

2. Preparation of IL-8, CRP monoclonal antibody-coated enzyme label plate: PBS (pH of 7.2-7.4) used for IL-8 monoclonal antibody is diluted to 2 g/ml, and added into odd rows of the enzyme label plate at 100 μl/well; PBS (pH of 7.2-7.4) used for CRP monoclonal antibody is diluted to 2 μg/ml, added into even rows of the enzyme label plate at 100μl/well, and coated at 2-8° C. for 16 hours; on the next day, the coating liquid is discarded and pat dry, the plates are washed with a washing liquid at 250 μl/well; the washing liquid is discarded, a confining liquid is added (1% BSA in PBST, pH at 7.2-7.4) at 200 μl/well and confined at 2-8° C. for 12 hours; on the next day, the confining liquid is discarded and pat dry, the plates are washed with a washing liquid for 3 times at 250 μl/well; the washing liquid is discarded and pat dry, packaged in aluminium foil bags and preserved at 2-8° C.

3. Preparation of HRP-labelled mouse-anti-cattle IL-8, CRP monoclonal antibody: Following the instruction of EZ-Link Activated Peroxides Antibody Labeling Kit (Thermo Scientific Pierce #31497), mouse-anti-cattle IL-8 and CRP monoclonal antibodies are labelled with HRP respectively. The labelled monoclonal antibodies are diluted with PBS, the optimum dilution is determined by a sandwich ELISA method. A commercial enzyme-labelled antibody stabilizer from Thermo Co. (Item No. 37548) is added to formulate a 100× enzyme-labelled antibody stock solution, which is filtered over a 0.22 μm filter membrane to remove bacteria, subpackaged in proper quantity aseptically, and preserved in dark at 2-8° C.

4. Preparation of standard: The recombinantly expressed IL-8 at a concentration of 1 μg/ml is diluted with a sample diluent (0.1% BSA in PBST, at pH 7.2-7.4) into 8 gradients (0, 1, 5, 10, 50, 100, 500, 1000 μg/ml), and formulated 15 min before detection. The recombinantly expressed CRP at a concentration of 1 μg/ml is diluted with a sample diluent (0.1% BSA in PBST, at pH 7.2-7.4) into 8 gradients (0, 6.25, 12.5, 25, 50, 100, 200, 400 ng/ml), and formulated 15 min before detection.

5. Adding samples: Plasma after PPD-B stimulation is thawed, mixed by inverting gently, and diluted with a sample diluent (0.1% BSA in PBST, at pH 7.2-7.4) by 5 folds, 8 folds and 200 folds. Taking monoclonal antibody-coated plates (according to the number of samples, the plates can be used separately), the first row is added with diluted IL-8 standard, the second row is added with diluted CRP standard, the third and the fourth rows are added with diluted samples at 100 μl/well, mixed sufficiently and scaled, and reacted in dark at room temperature (22-26° C.) for 60 minutes. The reaction plates are taken out, the reaction fluid is discarded, and each well is washed for 5 times by adding 250 μl 1×washing liquid, and pat dry at the last time. The operations of adding samples are shown as below:

TABLE 6
Schematic table showing adding samples

| | IL-8 | | CRP | | | |
|---|---|---|---|---|---|---|
| Samples | Standard | | Standard | | Samples | Samples |
| 1 | 1000 | pg/ml | 400 | ng/ml | 1-PPDB 20× | 1-PPDB 5× |
| 2 | 500 | pg/ml | 200 | ng/ml | 1-PPDB 200× | 1-PPDB 20× |
| 3 | 100 | pg/ml | 100 | ng/ml | 2-PPDB 20× | 2-PPDB 5× |
| 4 | 50 | pg/ml | 50 | ng/ml | 2-PPDB 200× | 2-PPDB 20× |
| 5 | 10 | pg/ml | 25 | ng/ml | 3-PPDB 20× | 3-PPDB 5× |
| 6 | 5 | pg/ml | 12.5 | ng/ml | 3-PPDB 200× | 3-PPDB 20× |
| 7 | 1 | pg/ml | 6.25 | ng/ml | Sample | Sample |
| | | | | | Diluent | Diluent |
| 8 | 0 | pg/ml | 0 | ng/ml | | |

6. Adding Enzyme-Labelled Antibody

100×HRP-anti-cattle IL-8 monoclonal antibody and 100×HRP-anti-cattle CRP monoclonal antibody are diluted with enzyme-labelled antibody diluent (0.1% BSA in PBST, at pH 7.2-7.4) by 100 times respectively. The odd rows are added with anti-IL-8 antibodies, and the even rows are added with anti-CRP antibodies at 100 μl/well, and reacted in dark at room temperature (22-26° C.) for 60 minutes. The reaction plates are taken out, the reaction fluid is discarded, and each well is washed for 5 times by adding 250 μl 1×washing liquid, and pat dry at the last time.

7. Colour-developing and stopping: A substrate colour-developing solution is added at 100 μl/well, and reacted in dark at room temperature (22-26° C.) for 30 minutes, with timing started from adding the first well. Following the sequence of adding the substrate colour-developing solution, 50 μl stop buffer is added into each well in turn, blended gently, and determined OD_{450 nm} values by a microplate reader within 10 minutes.

8. Data analysis: When OD_{450 nm} of the blank control is <0.3, the results are valid. The average value of blank control is subtracted from OD_{450 nm} readout of each well, with the difference between OD_{450 nm} of standard and the blank control as the horizontal ordinate, and with the concentration of standard as the vertical coordinate, a linear regression curve of standard is plotted. The concentration of sample is calculated according to the difference between OD_{450 nm} of the sample and the blank control, then multiplies by its dilution factor to be the concentration of IL-8 and CRP in plasma of this sample.

9. Establishment of diagnostic criteria: 21 PCR positive tuberculosis cattles (bTB PCR-P), 21 PCR negative tuberculosis cattles (bTB_{PCR-N}) and 19 tuberculosis negative cattles (NC) are screened according to the method of steps 1-5 in Embodiment 1. Heparin lithium anti-coagulant blood is harvested aseptically, and added onto a 24-well tissue culture plate at 1.5 ml per well. Each well is aseptically added with 100 μl PPD-B (300 IU/ml), blended with shaking gently and then incubated in a incubator at 37° C. and C02 for 20 to 24 hours. The upper plasma is sucked up carefully and transferred into a 1.5 ml centrifuge tube, and the concentration of IL-8 in plasma is detected with the IL-8, CRP sandwich ELISA detection kit of the invention. An ROC analysis is performed on the concentration of IL-8 in plasma of tuberculosis cattles (including PCR positive and negative, 42) and control cattles (19) after PPD-B stimulation, to evaluate the threshold of detection on tuberculosis cattles; an ROC analysis is performed on the concentration of CRP in plasma of tuberculosis PCR-positive cattles and PCR negative cattles after PPD-B stimulation, to evaluate the threshold of detection on tuberculosis cattles at the discharge period of bacteria.

TABLE 7
ROC Analysis

| | Area Under Curve | Sensitivity | 95% CI^{a} | Specificity | 95% CI^{a} | Threshold |
|---|---|---|---|---|---|---|
| Stimulus | (AUC) | (%) | (%) | (%) | (%) | (ng/ml) |
| PPD-B-induced | 0.9662 | 92.86 | 80.52-98.50 | 94.74 | 73.97-99.87 | >30 |
| IL-8 | | 92.86 | 80.52-98.50 | 100 | 82.35-100.0 | >42 |
| PPD-B-induced | 1.0000 | 100 | 83.89-100.0 | 95.24 | 76.18-99.88 | >700 |
| CKP | | 100 | 83.89-100.0 | 108 | 83.89-100.0 | >790 |

It is shown from the results that, under PPD-B specific stimulation, IL-8 is capable of differentiating tuberculosis positive cattles from negative cattles, when the detection specificity is 100%, its detection sensitivity can up to 96.62%, and the AUC is 0.9662. When differentiating tuberculosis cattles at the discharge period of bacteria from those not at the discharge period of bacteria, the AUC of CRP is 1, and when the specificity is 100%, the detection sensitivity can up to 100%. (Table 7).

TABLE 8
Variance analysis

| | | Tuberculosis | Tuberculosis | |
|---|---|---|---|---|
| | | cattles at | cattles not at | |
| | | the discharge | the discharge | |
| Detection | | period of | period of | Negative |
| Factor | Item | bacteria | bacteria | cattles |
| IFN-γ | Mean value | 7.330 | 7.731 | 0.329 |
| | Standard | 5.734 | 5.755 | 0.220 |
| | deviation | | | |
| IL-8 | Mean value | 144.195 | 125.834 | 18.163 |
| | Standard | 69.109 | 70.962 | 12.349 |
| | deviation | | | |
| CRP | Mean value | 1584.702 | 395.213 | 389.465 |
| | Standard | 536.931 | 191.179 | 162.747 |
| | deviation | | | |

Diagnostic criteria: To guarantee the detection specificity of tuberculosis cattles, according to the analysis results of ROC, the detection specificity is set at 100%, the sensitivity is higher than 90%, when the concentration of IL-8 in plasma after PPD-B stimulation is ≥42 ng/ml, it is determined as tuberculosis positive; when the concentration of IL-8 in plasma after PPD-B is <42 ng/ml, it is determined as tuberculosis negative. When the concentration of CRP in plasma of tuberculosis positive cattles after PPD-B stimulation is ≥790 ng/ml, it is determined as cattles at the discharge period of bacteria, while when the concentration of CRP in plasma after PPD-B stimulation is <790 ng/ml, it is determined as cattles not at the discharge period of bacteria. Different sample sizes may affect the accuracy of detection threshold, however, the average concentration of IL-8 in plasma of tuberculosis cattles after PPD-B stimulation is greater than 5-6 times of that in tuberculosis negative cattles, significantly higher than in negative cattles; the concentration of CRP in plasma of tuberculosis cattles at the discharge period of bacteria after PPD-B stimulation is greater than 3-4 times of those in tuberculosis cattles not at the discharge period of bacteria and in negative cattles, significantly higher than in tuberculosis cattles not at the discharge period of bacteria and in negative cattles. Therefore, the difference in sample quantity has little effect on the overall range of the threshold.

### Embodiment 5 Clinical Evaluation on the Bovine Tuberculosis IL-8, CRP Detection Kit of the Invention

At a cattle camp, 84 cattles are detected by the tuberculin skin test of Embodiment 1. 10 ml of venous blood is harvested from a cattle aseptically, and injected into a heparin lithium anticoagulant blood collection tube, which is inverted gently for 3-5 times, and sent back to the laboratory at room temperature for 20 hours. The anti-coagulant blood is added onto a 24-well tissue culture plate at 1.5 ml/well. Each well is aseptically added with purified bovine tuberculin (PPD-B), purified avian tuberculin (PPD-A), and PBS each 100 μl, blended with shaking and then incubated in a incubator at 37° C. and CO₂ for 20 to 24 hours. 200 μl of upper plasma is sucked up carefully and transferred into a 1.5 ml centrifuge tube, ready for use (the plasma can be stored at 2 to 8° C. for 7 days, and can be stored at −20° C. for several months). Following the instruction of bovine IFN-γ detection kit (purchased from Prionics Co.), PPD-B, PPD-A and PBS stimulation samples are detected. The concentrations of IL-8 and CRP in plasma after PPD-B stimulation are detected with the kit constructed in the Embodiment 4 of the invention. Following the nested PCR detection method used for the nasal swab secretion in Embodiment 1, nasal swabs are harvested from each cattle and detected.

TABLE 9

| | | IL-8, CRP Detection Kit | | | |
|---|---|---|---|---|---|
| | | for Bovine Tuberculosis | | | |
| | | Positive | | Negative | |
| Tuberculin skin test and | Positive | 34 | (A) | 1 | (C) |
| Interferon gamma release assay | Negative | 1 | (B) | 49 | (D) |

Sensitivity of IL-8, CRP detection method for Bovine tuberculosis relative to tuberculin skin test and interferon gamma release assay=A/(A+C)×100%=97%

Specificity of IL-8, CRP detection method for Bovine tuberculosis relative to tuberculin skin test and interferon gamma release assay=D/(B+D)×100%=98%

Coincidence rate between IL-8, CRP detection method for Bovine tuberculosis and tuberculin skin test and interferon gamma release assay=(A+D)/(A+B+C+D)×100%=97.65%

Positive Cattles: There are 35 cattles determined as double positive by detection with interferon gamma release assay and tuberculin skin test;

Negative Cattles: There are 50 cattles determined as double negative by detection with interferon gamma release assay and tuberculin skin test.

It is shown from the experimental results that: Coincidence rate between IL-8, CRP detection method for Bovine tuberculosis of the invention and the traditional interferon gamma release assay and tuberculin skin test can up to 97.65%, the sensitivity of detection can up to 97%, and the specificity reaches 98%. These test data indicate that the IL-8, CRP detection method for Bovine tuberculosis has high sensitivity and specificity, and can be used for differentiating tuberculosis positive cattles from negative cattles.

TABLE 10

| | | IL-8, CRP Detection Kit | | | |
|---|---|---|---|---|---|
| | | for Bovine Tuberculosis | | | |
| | | Positive | | Negative | |
| Nested PCR detection | Positive | 13 | (A) | 1 | (C) |
| for nasal swab secretion | Negative | 0 | (B) | 21 | (D) |

Sensitivity of IL-8, CRP detection method for Bovine tuberculosis relative to nested PCR detection for nasal swab secretion=A/(A+C)×100%=93%

Specificity of IL-8, CRP detection method for Bovine tuberculosis relative to nested PCR detection for nasal swab secretion=D/(B+D)×100%=100%

Coincidence rate between IL-8, CRP detection method for Bovine tuberculosis and nested PCR detection for nasal swab secretion=(A+D)/(A+B+C+D)×100%=97.14%

Positive Cattles: There are 35 cattles determined as double positive by detection with interferon gamma release assay and tuberculin skin test;

Tuberculosis cattles at the discharge period of bacteria: There are 14 cattles determined as positive by detection with nested PCR detection for nasal swab secretion;

Tuberculosis cattles not at the discharge period of bacteria: There are 21 cattles determined as positive by detection with nested PCR detection for nasal swab secretion.

It is shown from the experimental results that: Coincidence rate between IL-8, CRP detection method for Bovine tuberculosis and nested PCR detection for nasal swab secretion can up to 97.14%, the sensitivity of detection can up to 93%, and the specificity reaches 100%. These test data indicate that the IL-8, CRP detection method for Bovine tuberculosis has high sensitivity and specificity, and can be used for differentiating tuberculosis cattles at the discharge period of bacteria from tuberculosis cattles not at the discharge period of bacteria.

The above descriptions are only preferred embodiments of the invention. It should be noted to persons with ordinary skills in the art that several improvements and modifications can be made without deviating from the technical principle of the invention. These improvements and modifications should also be considered as the protection scope of the invention.

1. Diagnostic markers for Bovine tuberculosis, which are IL-8 and/or CRP.

2. A use of the diagnostic markers according to claim 1 in the preparation of diagnostic kits for Bovine tuberculosis.

3. The use according to claim 2, wherein when the concentration of IL-8 in plasma is ≥42 ng/ml, it is determined as Bovine tuberculosis positive; after PPD-B stimulation, when the concentration of IL-8 in plasma is <42 ng/ml, it is determined as Bovine tuberculosis negative.

4. The use according to claim 2, wherein when the concentration of IL-8 in plasma is greater than five times of that in tuberculosis negative cattles, it is determined as Bovine tuberculosis positive.

5. A use of the diagnostic markers according to claim 1 in the preparation of diagnostic kits for tuberculosis cattles at the discharge period of bacteria and tuberculosis cattles not at the discharge period of bacteria.

6. The use according to claim 5, wherein when the concentration of IL-8 in plasma is ≥42 ng/ml, it is determined as tuberculosis positive; when the concentration of IL-8 in plasma is <42 ng/ml, it is determined as tuberculosis negative;
when the concentration of CRP in plasma is ≥790 ng/ml, it is determined as tuberculosis positive cattles at the discharge period of bacteria, when the concentration of CRP in plasma is <790 ng/ml, it is determined as cattles not at the discharge period of bacteria;
when the concentration of IL-8 in plasma is ≥42 ng/ml, and the concentration of CRP in plasma is <790 ng/ml, it is determined as tuberculosis positive cattles not at the discharge period of bacteria.

7. The use according to claim 5, wherein the concentration of CRP in the plasma of tuberculosis cattles at the discharge period of bacteria is greater than 3 times of that in tuberculosis cattles not at the discharge period of bacteria and that in negative cattles.

8. The use according to claim 2, wherein the kits are ELISA kits.

9. The use according to claim 8, wherein the ELISA kits further contain stimulus, the stimulus is bovine tuberculin, avine tuberculin, CFP-10, ESAT-6 or PBS.

10. A diagnostic kit for tuberculosis cattles at the discharge period of bacteria and tuberculosis cattles not at the discharge period of bacteria, which contains detection reagents for detecting the expression level of IL-8 and/or CRP.

11. The use according to claim 4, wherein the kits are ELISA kits.

12. The use according to claim 6, wherein the kits are ELISA kits.

13. A use of the reagents for detecting the diagnostic markers according to claim 1 in the preparation of diagnostic kits for Bovine tuberculosis.

14. The use according to claim 13, wherein the kits are ELISA kits.

15. The use according to claim 13, wherein when the concentration of IL-8 in plasma is ≥42 ng/ml, it is determined as Bovine tuberculosis positive; after PPD-B stimulation, when the concentration of IL-8 in plasma is <42 ng/ml, it is determined as Bovine tuberculosis negative.

16. The use according to claim 15, wherein the kits are ELISA kits.

17. The use according to claim 13, wherein when the concentration of IL-8 in plasma is greater than five times of that in tuberculosis negative cattles, it is determined as Bovine tuberculosis positive.

18. A use of the reagents for detecting the diagnostic markers according to claim 1 in the preparation of diagnostic kits for tuberculosis cattles at the discharge period of bacteria and tuberculosis cattles not at the discharge period of bacteria.

19. The use according to claim 18, wherein when the concentration of IL-8 in plasma is ≥42 ng/ml, it is determined as tuberculosis positive; when the concentration of IL-8 in plasma is <42 ng/ml, it is determined as tuberculosis negative;
when the concentration of CRP in plasma is ≥790 ng/ml, it is determined as tuberculosis positive cattles at the discharge period of bacteria, when the concentration of CRP in plasma is <790 ng/ml, it is determined as cattles not at the discharge period of bacteria;
when the concentration of IL-8 in plasma is ≥42 ng/ml, and the concentration of CRP in plasma is <790 ng/ml, it is determined as tuberculosis positive cattles not at the discharge period of bacteria.

20. The use according to claim 18, wherein the concentration of CRP in the plasma of tuberculosis cattles at the discharge period of bacteria is greater than 3 times of that in tuberculosis cattles not at the discharge period of bacteria and that in negative cattles.
